(19) United States
(12) Patent Application Publication
(10) Pub. No.: US 2004/0029786 A1
(43) Pub. Date: Feb. 12, 2004

(54) Treatment of ulcerative colitis by introducing CEP antigen composition

(76) Inventor: Kiron M. Das, Baskin Ridge, NJ (US)

Correspondence Address: PERKINS COIE LLP, POST OFFICE BOX 1208, SEATTLE, WA, 98111-1208, US

(21) Appl. No.: 10/437,404

(22) Filed: May 8, 2003

Related U.S. Application Data
(60) Provisional application No. 60/379,379, filed on May 8, 2002.

(51) Int. Cl. A61K 38/17

(52) U.S. Cl. 514/8

(57) ABSTRACT

One aspect of the invention pertains to a method for treating ulcerative colitis and Crohn's disease by administering a composition comprising colonic epithelial protein (CEP) to induce tolerance. Another aspect of the invention provides for a composition comprising a colon epithelial cell extract, a LS-180 human colon cancer cell extract, a normal colon cell extract and T lymphocytes of a subject fed with the colon epithelial cell extract in the treatment of inflammatory bowel disease and/or inflammation. Yet another aspect of the invention is directed to methods for treating inflammatory bowel disease and/or inflammation by administering the aforementioned compositions to a subject.

### RELATED APPLICATION

This application claims the benefit of priority to U.S. Provisional Patent Application No. 60/379,379, filed May 8, 2002, which is hereby incorporated by reference in its entirety as if fully set forth herein.

### FIELD OF THE INVENTION

This invention relates to the field of gastroenterology. Specifically it relates to the treatment of inflammatory bowel disease and/or inflammation.

### BACKGROUND OF THE INVENTION

Inflammatory bowel disease (IBD) describes a number of gastrointestinal disorders. Of these diseases, Crohn's disease and ulcerative colitis are two of the main disease entities. Crohn's disease is a serious inflammatory disease of the gastrointestinal tract. It predominates in the ileum of the small intestine and the large intestine (colon), but may occur in any section of the gastrointestinal tract.

Ulcerative Colitis (UC) is an inflammation of the lining of the colon and rectum, particularly the mucosa. The disease typically begins in the rectosigmoid area extending proximally through the colon, and may eventually attack the majority of the large bowel resulting in acute and/or chronic inflammation. Acute and chronic inflammation within the colonic mucosa generates inflammatory mediators, called cytokines, which perpetuate the inflammatory process. UC is characterized by flare-ups followed by long periods of remissions. It is a lifelong disease that is estimated to affect as many as two million people in the United States. Symptoms of UC include increased stool frequency, bloody diarrhea, rectal bleeding, abdominal tenderness, cramping, weight loss, anemia, intestinal pain, and fever. Victims of UC are also at an increased risk for the development of intestinal cancer. The onset of UC may even cause psychological problems for some people, such as anxiety and depression, while under the strain of this debilitating disease.

Currently, there is a lack of a method for keeping UC in remission indefinitely. Present treatment involves the use of various medicating drugs including sulfonamide, sulfapyridine, salicylate, prednisone, hydrocortisone, and immunosuppressive drugs. Unfortunately, therapy with such drugs entails side effects including nausea, vomiting, heartburn, diarrhea, headaches, weight gain, acne, facial hair, hypertension, mood swings, increased risk of infection, and bone loss. Some people undergo diet modification as a less harsh alternative, but this is not entirely effective for most UC patients. In severe cases, surgery is necessary to remove a diseased colon, including a proctocolectomy with Brooke ileostomy or continent ileostomy, or an ileoanal anastomosis, both of which involve increased health risks. Accordingly, there is a need for additional treatments and for therapeutic compositions to treat UC.

### SUMMARY OF THE INVENTION

One aspect of the invention provides novel compositions for the treatment of inflammatory bowel disease (IBD) and/or inflammation. In one embodiment of the present invention, the compositions include acolonic epithelial protein (CEP), human colon tumor cell extract, normal colon cell extract, or T lymphocytes from individuals fed with colonic antigen extract (either individually or as a mixture thereof). The compositions of At least one aspect of the invention are capable of inducing oral tolerance to treat IBD. CEP alone appears to complex with a p40 colonic autoantigen or a related tropomyosin molecule. Particularly, CEP is a glycoprotein reactive to the monoclonal antibody 7E₁₂H₁₂. Das et al., Production and Characterization of Monoclonal Antibodies to a Human Colonic Antigen Associated with Ulcerative Colitis Cellular Localization of the Antigen by Using the Monoclonal Antibody, J. Immuno. Vol. 139: 77-84.

Another aspect of the present invention provides methods for treating IBD and/or inflammation comprising the step of administering to a subject CEP, a human colon tumor cell extract, a normal colon cell extract, or T lymphocytes from individuals fed with colonic antigen extract. The inventor's other issued patents and patent applications related to various aspects of gastric disorders including U.S. Pat. Nos. 5,869,048 and 5,888,743; as well as Ser. Nos. 09/046,049, 09/512,515 and 09/779,689 are incorporated by reference herein. A method for treating inflammatory bowel disease by administering to a subject a composition of an antigen effective to stimulate tolerance is described in this disclosure.

Yet another aspect of the present invention concerns a colon epithelial protein, which has been found to reduce inflammation in a rat model in which an induced colitis has been affected. The method of treatment disclosed in this application achieves the goal of inducing antigen-specific tolerance, thereby minimizing the risk that is typically associated with immunosuppressive therapy.

In one embodiment, the therapeutic composition of pertains to administering a therapeutically effective composition of the CEP to a subject. CEP is preferably from the colon region because the antigen inducing oral tolerance to colitis is organ specific.

In another embodiment, tolerance, more specifically oral tolerance, may be induced by the passive transfer of T lymphocytes from individuals fed with colonic antigen extract. Although administration of the therapeutic composition is preferably by oral means, the composition, or fragments thereof, may be administered parenterally, enterally, and by other means known to those skilled in the art. Furthermore, the composition may be administered in liquid, solid, or semi-solid forms. The compositions of At least one aspect of the invention may also be used for the purpose of reducing inflammation, specifically inflammation of the GI tract and in particular inflammation of the colon.

### BRIEF DESCRIPTION OF THE DRAWINGS

A better understanding of this invention can be obtained when the following detailed description of the preferred embodiments is considered in conjunction with the following drawings.

FIG. 1A. Photograph of gross appearance of rat colonic mucosa from the distal colon in a rat that received rectal trinitrobenzene sulfonic acid (“TNBS”). Severe inflammation is evident of the rectosigmoid colon, including large ulcer, edema, thickening of the wall, and adhesion.

FIG. 1B. Photograph of gross appearance of rat colonic mucosa from the distal colon in a rat that received LS-180 human colon cell extract, followed by rectally administered TNBS. No inflammatory change is present due to protection by the LS-180 human colon cell extract.

FIG. 1C. Photograph of gross appearance of rat colonic mucosa from the distal colon in a rat that received HT-1080 human fibroblast cell extract, followed by rectally administered TNBS. Severe inflammation is apparent including mucosal ulceration, edema, and thickening of the wall.

FIG. 2A. Photograph displaying the histology of rectosigmoid colon tissue in a rat that received rectal TNBS. Severe transmural inflammation is evident.

FIG. 2B. Photograph displaying the histology of rectosigmoid colon tissue in a rat that received LS-180 human colon cell extract, followed by rectally administered TNBS. There is no inflammation and the tissue is normal.

FIG. 2C. Photograph displaying the histology of rectosigmoid colon tissue in a rat that received HT-1080 human fibroblast cell extract, followed by rectally administered TNBS. Severe transmural inflammation is apparent.

FIG. 2D. Photograph of gross appearance of rat colonic mucosa from the distal colon in a rat that received T lymphocytes from mesenteric lymph nodes of rats that were fed LS-180 cell extract, followed by rectally administered TNBS. There are minimal inflammatory changes due to the protection after transfer of T lymphocytes.

FIG. 3. Histogram showing wet weight of the distal colon (0 to 8 cm) in different groups of rats. A heavier distal colon indicates increased inflammation and lesser protection against the disease. The weight of the colons of rats given only rectal TNBS (RE-TNBS group) was significantly higher than that in the controls. The weight of the distal colon from animals that were fed HT-1080 cell extract (HTE-TNBS group) or normal rat small intestine extract (NSE-TNBS group) was similar to the rats only administered TNBS. The weight of the colon from rats fed LS-180 (LSE-TNBS group) and normal rat colon extract (NCE-TNBS group) had significantly lower weight of the distal colon from rats only administered TNBS. Rats that received T lymphocytes from mesenteric lymph nodes (LS-L-TNBS group) or spleen (LS-S-TNBS) showed weights significantly lower than rats of the RE-TNBS group.

FIG. 4. Histogram showing the disease scores on a scale from 0 to 5, where 5 is most severe of the different groups of rats. A higher disease score indicates less protection against the disease. The rats administered only TNBS (RE-TNBS group) had a high mean score of 4.5, indicating significant disease. Rats of the HTE-TNBS and NSE-TNBS groups also had significant disease. Rats administered LS-180 cell extract (LSE-TNBS group), normal rat colon extract (NCE-TNBS group), T lymphocytes from rats fed LS-180 extract (LS-L-TNBS and LS-S-TNBS groups) indicated low disease scores, similar to the control group.

FIG. 5. Histogram showing the thickness of the distal colon wall from luminal surface to serosa in the different groups of rats. Increased thickness of the distal colon wall indicates the spread of disease. The thickness of the distal colon wall is measured in micrograms. When compared to the control group, the thickness of the distal colon wall significantly increased in the RE-TNBS, HTE-TNBS, and NSE-TNBS groups. In the LSE-TNBS, NCE-TNBS, LS-L-TNBS, and LS-S-TNBS groups, the thickness of the distal colon wall was significantly decreased.

FIG. 6. Histogram showing MPO (myeloperoxidase) activity in the different groups of rats measured in-the distal colon, proximal colon, and small intestine. MPO activity is measured in MPO units (10⁻³) per milligram of total tissue protein from the distal colon. MPO activity is an objective measure of inflammatory cellular infiltrate and lower MPO activity indicates increased protection. Low MPO activity was seen in all groups except RE-TNBS, NSE-TNBS, and HTE-TNBS.

FIG. 7. Histogram showing serum concentration of cytokine IFN-γ in different groups of rats. Stimulated cells from inflamed tissue produce increased amounts IFN-γ. There was a significant increase in the serum IFN-γ levels in the RE-TNBS, NSE-TNBS, and HTE-TNBS groups when compared with the control group, indicating severe inflammation. As for the LSE-TNBS, NCE-TNBS, LS-L-TNBS, and LS-S-TNBS groups, oral tolerance was indicated because of the low levels of IFN-γ serum.

FIG. 8. Histogram showing serum concentration of cytokine TGF-β in different groups of rats. High levels of TGF-β serum demonstrate the development of tolerance due to a Th3 type of immune response to inflammation. The LSE-TNBS, NCE-TNBS, LS-L-TNBS, and LS-S-TNBS groups show high TGF-β levels, whereas the RE-TNBS, NSE-TNBS, and HTE-TNBS groups have lower levels of TGF-β serum. Oral antigen present in LS-180 extract and normal rat colon induces TGF-β to decrease inflammation.

### DETAILED DESCRIPTION OF THE INVENTION

There is considerable evidence that UC is an autoimmune disease wherein inflammatory cells that normally defend the body against antigens are unable to distinguish between foreign substances and the body's own tissue. Consequently, the inflammatory cells that target specific autoantigens attack healthy tissue, causing chronic inflammation.

Autoimmunity has been implicated in the pathogenesis of UC since its initial report 36 years ago, which demonstrated the presence of circulating antibody in UC against colonic antigen (Perlman, P., J. Exp. Med. 110:657-673 (1959)). Previous studies involving treatment of colitis have demonstrated the presence of colonic autoantigen and the antibodies reactive against this protein in UC (see, for example, Takahasi et al., J. Clin. Invest. 76:311 (1985) and Das et al., Gut 33:48 (1992)). Das, U.S. Pat. No. 5,869,048 describes treatment by administering an antibody to bind to a colonic antigen associated with UC.

In addition to treating UC with antibodies, colon inflammation may also be reduced by orally administering autoantigens. This phenomenon, known as oral tolerance, restores the body's ability to tolerate protein antigens. Oral tolerance induces immunological hyporesponsiveness toward specific protein antigens causing the secretion of immunosuppressive cytokines. Two known processes to mediate oral tolerance are first, the induction of anergy, resulting in functional inactivation and/or deletion of antigen-specific T cells; and secondly, active suppression after multiple low-dose antigen feeding. (Lee et al., Am. J. of Gastroenterol. 95:861-2 (2000)). Oral tolerance has been used for the treatment of various autoimmune diseases in animals, and recently in human beings as well (Weiner et al., Science 259:1321-4 (1993)).

It is known that administration of antigens may induce oral tolerance (McGuinness et al., U.S. Pat. No. 5,948,407). For example, a recent study regarding autoimmune myasthenia gravis determined that AChR (acetylcholine receptor) effectively induced oral tolerance in mice and rats (Im et al., J. Clin Invest. 104:1723-30 (1999)). In the study, a recombinant fragment of AChR successfully treated the disease, and the AchR fragment-induced lymphocytes adoptively transferred disease suppression to other animals. Further, Braun et al., U.S. Pat. No. 6,033,864, describes a method for inducing tolerance in a pANCA-positive (pennuclear anti-neutrophil cytoplasmic antibodies) UC patient using a histone HI-like antigen, a porin antigen, and a Bacteroides antigen. The '864 patent teaches that the pANCA autoantibody present in the sera of most patients with UC reacts with histone H1. Verdu et al., in Clin. Exp. Immunol. 120:46-50 (2000), has also shown that orally administering antigens from intestinal flora anaerobic bacteria reduces colitis in mice.

Other recent studies have discovered the importance of colonic proteins in the development of oral tolerance for subjects with UC. Neurath et al., J. Exp. Med. 183:2605-16 (1996) used TNBS-haptenzed colonic proteins to stimulate oral tolerance in mice with TNBS-induced colitis. The mice were fed with haptenized colonic proteins, which prevent weight loss in mice given CD4+ lamina propria T cells from colitic mice. Although Neurath demonstrated that previously activated T cells were down regulated, the mice did not have ongoing colitis.

Another study has described feeding colitis-extracted proteins to rats to test whether oral tolerance is induced (Ilan et al., Am. J. of Gastroenterol. 95:966-973 (2000)). The study administered colitis-extracted proteins or bovine serum albumin (BSA) to rats with TNBS-induced colitis and colitis assessment was performed. Serum TGF-β and IFN-γ levels were also measured. Based on the degree of colonic ulceration, wall thickness, and serum levels, the investigators concluded that feeding colonic extract proteins induced suppressor T cells that mediate tolerance to tissue antigens.

Although previous studies have found that bacterial antigens may induce oral tolerance, at least one aspect of this invention determines that the colon epithelial antigen is organ specific and responsible for oral tolerance in colonic inflammation. At least one aspect of the invention is based on the discovery that specific proteins, CEP, may be involved in oral tolerance. Administration of CEP is an effective means of suppressing T-cell mediated, T-cell dependent immune reactions, or other immune reactions. The method of reducing an immune response is one that induces tolerance to administration of the antigen by inducing a suppressor T cell response to the administered antigen. By using rats with induced colitis, this invention demonstrates the role of host colon epithelial antigen in oral tolerance of UC. Another aspect of the invention pertains to the discovery that CEP is directly linked to suppressing UC. UC, generally accepted to be an autoimmune disease, is capable of being treated by inducing oral tolerance. CEP plays a critical role within the mucosal immune system to induce oral tolerance. By employing the method as disclosed herein, it was discovered that CEP is capable of inducing oral tolerance in induced rat modes. Prepared cell extracts, including human colon cancer cells, human fibroblast cells, homologous normal rat colon and rat small intestine were orally introduced to different groups of rats. T lymphocytes from mesenteric lymph nodes and spleen from rats fed with LS-180 human colon cancer cell extract and HT-1080 fibroblast extract were also orally administered. The rats were then rectally challenged with TNBS. The rats were subsequently killed in order to assess colonic inflammation and damage, and to determine the levels of tolerance in the antigen-treated rats. Cytokine levels were also examined to determine levels of protection and tolerance to UC.

Another aspect of the invention describes methods for treating inflammatory bowel disease by administering a therapeutically effective composition of CEP, human colon tumor cell extract (specifically LS-180 cells), normal colon epithelial cell extract and of T lymphocytes from individuals fed with colonic antigen extract, to a subject in need thereof. When treating IBD, specifically Crohn's disease and UC, the CEP can be in the form of LS-180 colon cell extract. Alternatively, oral tolerance may be passively transferred by T lymphocytes extracted from a subject fed a colonic antigen.

Another aspect of the invention therefore involves an immunological tolerance-inducing composition including CEP (or a fragment thereof), human colon tumor cell extract (specifically LS-180 cells), normal colon epithelial cell extract and of T-lymphocytes from individuals fed with colonic antigen extract (either individually or a mixture thereof). The CEP protein may be a number of colonic antigens, including a glycoprotein or tropomyosin molecule.

The therapeutic component of one aspect of this invention involves administering a composition of a protein and or proteins described above, to a subject to reduce inflammation caused by UC and/or Crohn's disease and the like. Preferably, the composition should be orally administered in various forms to a patient in such way that it is present at a sufficient concentration to adequately provide protection.

Although specific embodiments are described below, it should be understood that such embodiments are examples that are merely illustrative of a small number of the many possible specific embodiments that can represent applications of the principles of At least one aspect of the invention. Various modifications pertaining to the invention obvious to one skilled in the art are within the spirit, scope, and contemplation of At least one aspect of the invention.

“Inflammatory bowel disease,” otherwise referred to as “IBD,” is a chronic inflammatory condition of the digestive tract. UC and Crohn's disease are forms of IBD. UC involves inflammation of the inner lining of the colon and rectum. Crohn's disease may involve inflammation anywhere in the digestive tract from the mouth to the rectum.

As used herein the term “ulcerative colitis” means a disease that manifests itself as chronic, nonspecific ulcerations in the colon. Generally, features that may be present in UC include inflammation extending proximally from the rectum including and complicated by shallow ulcerations. The pathogenesis of UC, although not clearly understood, involves immune-mediated tissue damage. UC can represent a process of immune dysfunction directed against intrinsic intestinal mucosa cells and can occur in a mucosal site interfacing with the intestinal lumen.

The term “autoimmune” means an immune response directed at one's own tissues. Specifically, autoimmune disease is a malfunction of the immune system of mammals, in which the immune system fails to distinguish between foreign substances within the mammal and/or autologous tissues or substances. As a result, the immune system treats autologous tissues and substances as if they were foreign, and mounts an immune response against the autologous tissues.

The term “treatment” means both the prophylactic measures to prevent autoimmune diseases and the suppression or alleviation of symptoms after the onset of autoimmune diseases.

The term “antigen” refers to a substance that elicits an immune response. An “autoantigen” is any substance normally found within a subject that, in an abnormal situation, is no longer recognized by the lymphocytes or antibodies as part of the animal itself, and is the therefore attacked by the immunoregulatory system as though it were a foreign substance.

The term “CEP” is synonymous with CEP (including NCE (colonic extract) and LSE (LS-180 cell extract) proteins) and describes a glycoprotein (ME>200 kd) reactive to the monoclonal antibody 7E₁₂H₁₂ (I9M isotype). Das et al., Production and Characterization of Monoclonal Antibodies to a Human Colonic Antigen Associated with Ulcerative Colitis Cellular Localization of the Antigen by Using the Monoclonal Antibody, J. Immuno. Vol. 139: 77-84. The CEP may be haptenized or non-haptenized. “Epithelial” or “epithelium” means a cell sheet that covers an external surface or lines a cavity.

The term “antibody” means molecules, which may be polyclonal or monoclonal of any isotype, which respond to an antigen. An “autoantibody” is an antibody that reacts against one's own body. Specifically, “monoclonal antibody” refers to a population of antibody molecules that contain only one species of idiotope capable of binding a particular antigen epitope.

The term “hapten” means any molecule capable of being bound by an antibody.

The term “distal,” as used in “distal colon” of a rat means farthest from the center.

As used herein, “control” means the control group of rats that received no treatment, “RE-TNBS” means rectally administered trinitrobenzene sulfonic acid, “NCE-TNBS” means orally administered homologous normal rat colon followed by rectally administered TNBS, “NSE-TNBS” means orally administered rat small intestine followed by rectally administered TNBS, “LSE-TNBS” means orally administered tissue-extracted proteins in PBS from LS-180 human colon cancer cells followed by rectally administered TNBS, “HTE-TNBS” means orally administered tissue-extracted proteins in PBS from HT-1080 human fibroblast cells followed by rectally administered TNBS, “LS-L-TNBS” means injected T lymphocytes from mesenteric lymph nodes of rats fed with LS-180 human colon cancer cells followed by rectally administered TNBS, and “LS-S-TNBS” means injected T lymphocytes from the spleen of rats fed with LS-180 human colon cancer cells followed by rectally administered TNBS.

The term “fragment” means any polypeptide subset of that molecule capable of inducing tolerance.

The terms “introduction” or “administration” mean that the antigen, extract, or fragment thereof, is introduced orally, enterally, intranasaly, peritoneally, subcutaneously, transdermally, intratumorally, rectally, intra-arterially, intramuscularly, by rapid infusion, nasopharyngeal absorption, intranasopharangeally, or by dermoabsorption. “Oral administration” or “oral introduction” includes administration by way of the mouth.

The term “therapeutically effective amount” is an amount sufficient to achieve a desired biological effect. The amount will vary depending on a multiple of variables such as the individual's condition, age, sex, extent of disease, and other variables known to those of ordinary skill in the art.

The term “individual” refers to any individual animal capable of having IBD, including a human, non-human primate, rabbit, rat or mouse.

As used herein, “cytokine” is a molecule that is secreted for regulation of other cells. Cytokines act as mediators in autocrine, paracrine or intacrine fashion, as in the generation of an inflammatory or immune response. Cytokines are small to medium-sized (usually less than 30 kDa), hormone-like proteins governing numerous biological responses such as cell growth, cell maturation, and defense against infection.

Specifically, the cytokine “TGF-β” means transforming growth factor-beta and is a pro-inflammatory cytokine. The cytokine “IFN-γ” means interferon-gamma and is an anti-inflammatory cytokine.

Colonic Antigen Effect on Chronic Inflammation

At least one aspect of the invention is directed to the composition and use of a colonic antigen for treatment of inflammatory bowel disease. Specifically, the method comprises orally administering to a subject with IBD, specifically UC a therapeutically effective composition of epithelial cellular protein to induce oral tolerance. It is disclosed herein that by feeding CEP to rat models, the inflammation associated with the disease is greatly minimized because of the development of oral tolerance.

To determine the effect of colonic antigen on reducing colitis inflammation, colon cell extract, or colonic extract, was fed to selected groups of rats. The colonic extract was introduced as either human colon cancer cells LS-180 extract (LSE-TNBS group) or as normal rat colon extract (NCE-TNBS group). The other groups of rats received either no additional treatment (RE-TNBS group), HT-1080 human fibroblast cells (HTE-TNBS group) or rat small intestine (NSE-TNBS). Additionally, selected rats were injected with mesenteric lymph nodes (LS-L-TNBS group) and spleen (LS-S-TNBS group) removed from rats fed with LS-180 cell extract. Subsequently, all rats except the control group were rectally administered TNBS. Table 1 sets forth in detail the different groups of rats and corresponding steps of treatment for each group.

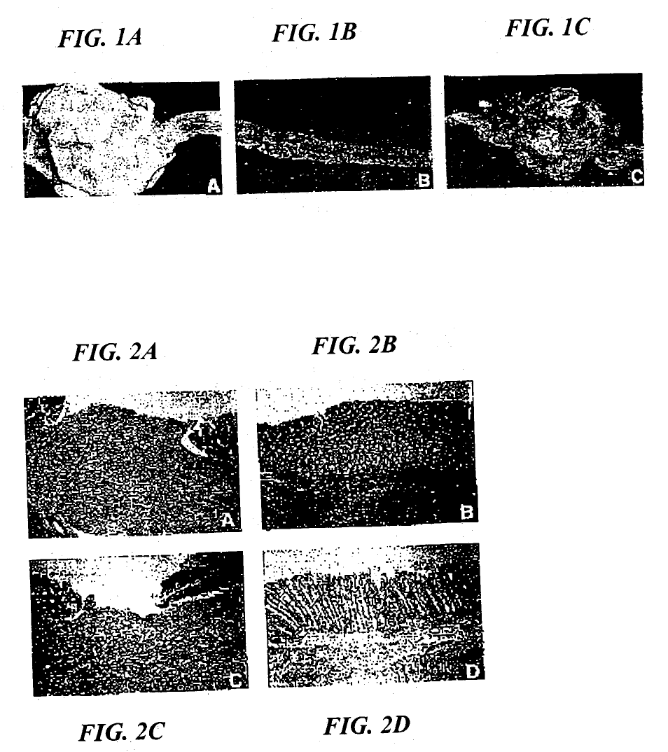

All rats were killed in order to assess the damage or by inflammation, or the lack thereof. FIGS. 1A-C and 2A-D exhibit the differences in inflammation of the rectosigmoid colon in rats that received only TNBS, rats that received human colon extract, and rats that received HT-1080 human fibroblast cells. Based on the criteria for scoring gross morphologic damage developed by Morris et al., Gastroenterol. 96:795-803 (1989) (Table 2), the rats in each group were assigned a disease score, as depicted in Table 3. Mortality rates were further indication of which rats effectively developed tolerance to the disease to survive, also listed in Table 3.

TABLE 1
Inducing oral tolerance in rats in different treatment groups.

| | Number | | |
|---|---|---|---|
| Groups | of rats | Treatment Procedures Listed in Sequential Steps | |
| Control | 4 | No treatment | |
| RE-TNBS | 22 | 30 mg/rat of TNBS in 50% ethanol, given intra- | |
| | | rectally | |
| NCE- | 6 | 1) | Colonic extract, 500 μg/rat was given by mouth |
| TNBS | | | on alternate days 5 times |
| | | 2) | 7 days rest |
| | | 3) | Intra-rectal TNBS (30 mg/rat in 50% ethanol) |
| NSE- | 6 | 1) | Small intestine extract, 500 μg/rat was given |
| TNBS | | | by mouth on alternate days 5 times |
| | | 2) | 7 days rest |
| | | 3) | Intra-rectal TNBS (30 mg/rat in 50% ethanol) |
| LSE- | 18 | 1) | LS-180 cell extract, 500 μg/rat was given by |
| TNBS | | | mouth on alternate days 5 times |
| | | 2) | 7 days rest |
| | | 3) | Intra-rectal TNBS (30 mg/rat in 50% ethanol) |
| HTE- | 6 | 1) | HT-1080 cell extract, 500 μg/rat was given by |
| TNBS | | | mouth on alternate days 5 times |
| | | 2) | 7 days rest |
| | | 3) | Intra-rectal TNBS (30 mg/rat in 50% ethanol) |
| LS-L- | 6 | 1) | 25 × 10⁴ T lymphocytes from mesenteric and |
| TNBS | | | iliac lymph nodes of rats fed LS-180 extract, |
| | | | 500 μg/rat was given by mouth on alternate |
| | | | days 5 times |
| | | 2) | Injected through tail vein into naive rats |
| | | 3) | 7 days rest |
| | | 4) | Intra-rectal TNBS (30 mg/rat in 50% ethanol) |
| LS-S- | 6 | 1) | 25 × 10⁴ T lymphocytes from spleen of rats |
| TNBS | | | fed LS-180 extract, 500 μg/rat was given by |
| | | | mouth on alternate days 5 times |
| | | 2) | Injected through tail vein into naive rats |
| | | 3) | 7 days rest |
| | | 4) | Intra-rectal TNBS (30 mg/rat in 50% ethanol) |
| | | | Table |

TABLE 2
Criteria for scoring of gross morphologic damages of rat tissue.

| Score | Gross Morphology |
|---|---|
| 0 | No damage |
| 1 | Localized hyperemia, but no ulcers |
| 2 | Linear ulcers with no significant inflammation |
| 3 | Linear ulcers with inflammation at one site |
| 4 | Two or more sites of ulceration and/or inflammation |
| 5 | Two or more major sites of inflammation and ulceration |
| | extending >1 cm along the length of the colon |

TABLE 3
Disease scores and mortality rates in different treatment groups.

| | Number of | | | | | | | |
|---|---|---|---|---|---|---|---|---|
| | rats | | | | | | | |
| Groups | Total | Dead | Number of rats affected with colitis | | | | | |
| Gross | | | 0 | 1 | 2 | 3 | 4 | 5 |
| Morphologic | | | | | | | | |
| Damage Scores | | | | | | | | |
| Control | 4 | 0 | 4 | 0 | 0 | 0 | 0 | 0 |
| RE-TNBS | 22 | 6 | 0 | 0 | 0 | 0 | 0 | 10 |
| NCE-TNBS | 6 | 6 | 2 | 1 | 2 | 1 | 0 | 0 |
| NSE-TNBS | 6 | 0 | 0 | 0 | 0 | 0 | 1 | 5 |
| LSE-TNBS | 18 | 1 | 1 | 7 | 5 | 2 | 0 | 2 |
| HTE-TNBS | 6 | 0 | 0 | 0 | 0 | 1 | 1 | 4 |
| LS-L-TNBS | 6 | 0 | 3 | 0 | 2 | 1 | 0 | 0 |
| LS-S-TNBS | 6 | 0 | 0 | 2 | 2 | 0 | 1 | 1 |

As determined in previous studies, feeding animals with extract of diseased colon tissue from animals with developed colitis results in suppression of TNBS-induced colitis. (Ilan et al., Am. J Gastroenterol. 95:966-73 (2000)). The tolerance induced by haptenized colon extract may be due to the hapten itself (Elson, J. Immunol. 157:2174-85 (1996)), colonic bacterial proteins (Sartor, Eur. J. Clin Invest. 28:1027-9 (1998)), or both. At least one aspect of the invention, however, indicates that both haptenization of the colonic extracted proteins is non-hapenized colonic extract may be used for inducing tolerance. Both the extract from non-haptenized colon epithelial cells and crude colonic extract from rat colon (groups LS-TNBS and NCE-TNBS, respectively) indicated low disease scores and overall protection (Table 3). Thus, the tolerance induced by LS-180 cell extract indicates that non-bacterial antigen(s) are capable of oral tolerance.

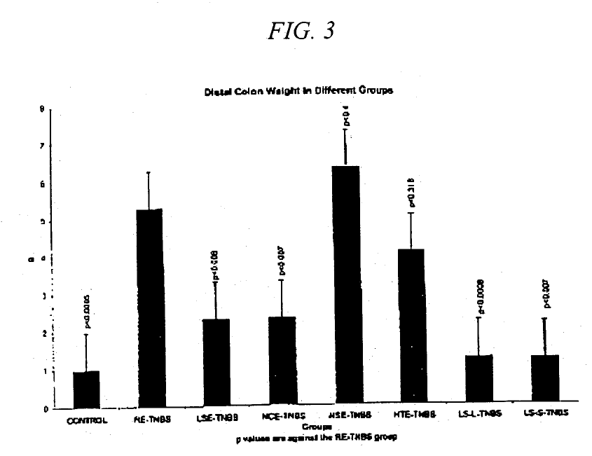

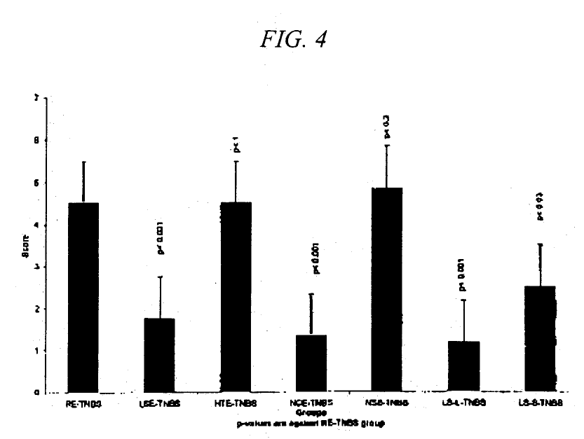

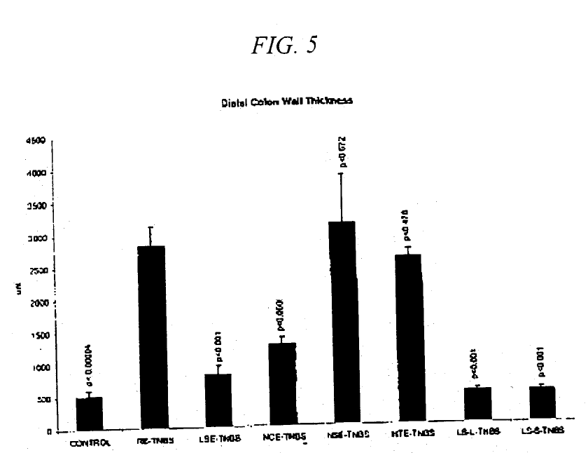

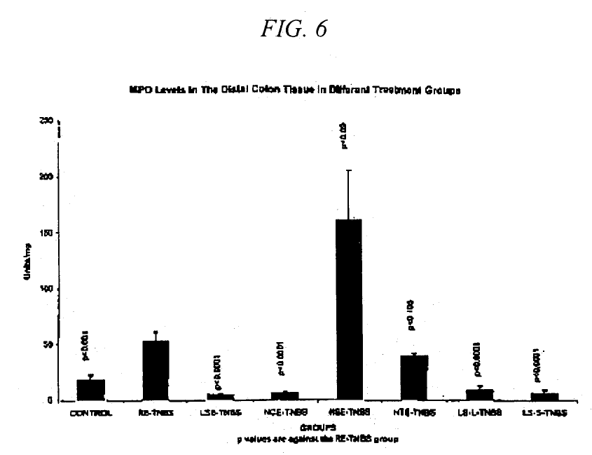

At least one aspect of the invention also shows that CEP clearly reduces inflammation in rats, particularly when compared with rats administered no colonic antigen. In a TNBS-induced model of colitis, the mortality rate of rats fed with human LS-180 extract compared with rats not fed with the colon epithelial cell extract was significantly lower (5% and 26%, respectively), as indicated in Table 3. The rats fed with the extract also had lower disease scores than the rats not fed the extract, thus indicating little or no inflammation of the colon (FIG. 4). Further, rats in the LSE-TNBS group had a lower weight of the distal colon (in grams) (FIG. 3), thinner distal colon walls (in micrograms) (FIG. 5), and less MPO activity (FIG. 6). These results were reproducible showing there is clear protection in animals fed with the LS-180 cell extracts. It is also determined that mucosal tolerance can be developed by orally administering CEP, the invention further demonstrates that CEP induced tolerance is colon specific. Rat colon extract is shown to induce oral tolerance, indicated by the low disease score and decreased mortality rate in Table 3 and FIG. 4. Similar to rats fed LS-180 extract, rats fed homologous normal rat colon (NCE-TNBS group) showed decreased inflammation of the colon when compared to rats of the RE-TNBS group (FIGS. 3, 5, and 6).

On the other hand, rats administered human fibroblast cell extract (HTE-TNBS group) had data comparable to that of the RE-TNBS group that experienced severe inflammation (Table 3, FIGS. 3-6). Oral tolerance, as evidenced by significant reduction of intestinal inflammation, is induced with human colon epithelial cells, but not human fibroblast cells. Additionally, rats fed with rat small intestine extract (NSE-TNBS group) showed higher mortality rates, higher disease scores, inflammation of the rat colon, thickness of colon walls, heavier colons and increased MPO activity. (Table 3, FIGS. 3-6). Both groups had similar data to that of the RE-TNBS group of rats, in which no additional treatment was administered. Therefore, oral tolerance is organ (colon) specific because protection is induced with rat colon extract, but not with human fibroblast cellular extract or rat small intestine extract. If UC falls into an organ-specific category, then the immune response is directed against antigen(s) associated with the target organ being damaged. The organ specificity of this invention may result from homing of regulatory lymphocytes to the correct bowel region.

Suppression of inflammation mediated by particular lymphocytes also demonstrates that CEP induces oral tolerance and organ specific protection. Specifically, there may be a passive transfer of oral tolerance by T lymphocytes. The T lymphocytes in rats fed with human colon epithelial cells (LS-180 extract) are capable of down-regulating the development of colitis (FIGS. 2D, 3, 5, and 6). The similar transfer of T lymphocytes from rats fed HT-1080 cell extract did not show any tolerance, thus, indicating that tolerance is organ specific. The combined data shows that oral tolerance is induced by a colon epithelium-specific antigen without haptenization, and this tolerance is likely generated by regulatory T cells present in the mesenteric lymph nodes and spleen.

Immune oral tolerance is further shown by the generation and balancing of pro-inflammatory and anti-inflammatory cytokines. Current research indicates that a class of proteins known as cytokines, products of immune-competent cells including macrophages, T-lymphocytes, and endothelial cells, are crucial in the initiation and maintenance of inflammation. Recently, it has become clear that what determines the characteristics of the intestinal inflammatory response is dependent on the cytokines involved during the response. A Th1 response, with an increased production of IFN-γ, and other pro-inflammatory cytokines, occurs when infected by inflammatory bowel diseases. A Th2 response is characteristic of the allergic subject, and there is some evidence that it is the predominant response in subjects with UC. More recently it has become clear that the intestinal mucosa has a unique subset of CD4⁺ T cells that secrete TGF-Li (Th3 cells) that provide help for IgA. These cells have down regulatory properties, and therefore, play an important role in the active suppression of oral tolerance and IgE response.

Depending upon cell type or conditions, the secretion of TGF-β can be induced by a number of different stimuli including steroids, retinues, EGG, TGF, vitamin D3, ILK, and activators of lymphocytes. TGF-β inhibits the proliferation of T-lymphocytes by down-regulating predominantly ILK-mediated proliferative signals. Oral antigen preferentially induces TGF-β because it serves as a switch factor for IgA in the intestine. TGF-β has been implicated as a mediator of a range of inflammatory disorders such as rheumatoid arthritis and nephritis, myelofibrosis, scleroderma, and pulmonary fibrosis.

A reciprocal relationship exists for IFN-γ levels in the serum. IFN-γ is produced mainly by T-cells and natural killer cells activated by antigens, mitogens, or alloantigens. It is produced by lymphocytes expressing the surface antigens CD4 and CD8. T cells with CD4 marker induce immune responses, and CD4 cells can be subdivided primarily as Th1 or Th2 cells. Secretion of IFN-γ contributes to an increase in mucosal permeability in IBD models, as described in Neurath et al., Immunol. Today 18:61-4 (1997).

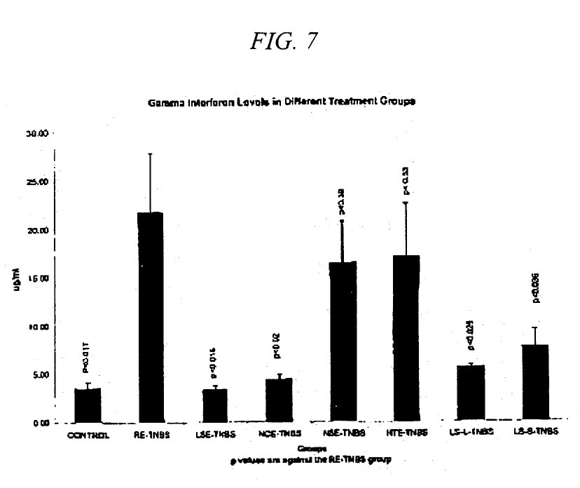

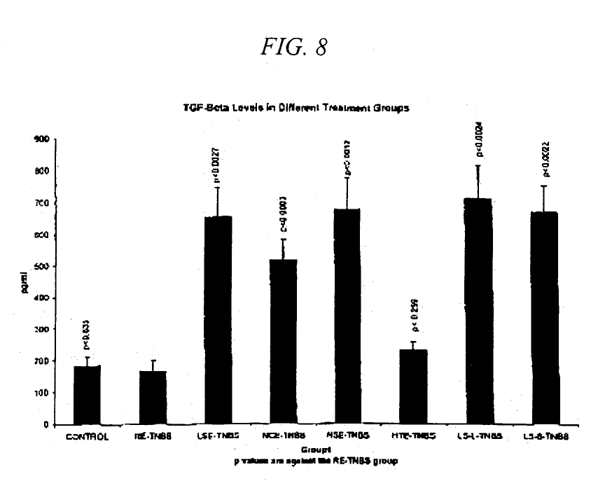

In at least one aspect of the invention, CEP is shown to be an effective tolerance-inducing protein, indicated by the opposing serum levels of the cytokines, TGF-βand IFN-γ. The significant increase in TGF-β levels in the serum of rats fed LS-180 cell extract and normal rat colon extract indicates that the introduction of CEP resulted in a Th3 type of immune response (FIGS. 7 and 8). High TGF-β levels were also found in rats that received mesenteric and splenic T lymphocytes from LS-180 extract fed rats (FIGS. 7 and 8).

Furthermore, rats fed with LS-180 extract experienced a significant decrease in IFN-γ levels, whereas the rats administered solely with TNBS had significantly higher levels of IFN-γ (FIGS. 7 and 8). The HTE-TNBS and NSE-TNBS groups also showed an increase in IFN-γ levels, which indicates significant colitis and no developed tolerance.

Protective Colonic Epithelial Antigen

One aspect of the invention also provides a tolerance-inducing composition for treating individuals suffering from UC comprising either individually, or as a mixture thereof, a CEP, a human colon tumor cell extract (specifically LS-180 cells), a normal colon epithelial cell extract or T lymphocytes from individuals fed with colonic antigen extract. With respect to CEP, it may be fragmented to produce tolerogenic fragments useful in accordance with treating UC patients. Additionally, purified CEP may be analyzed to determine its amino acid sequence. The availability of such sequence information allows for the production of CEP and its fragments by synthetic chemical techniques known to those skilled in the art.

The specific colonic epithelial antigen(s) involved in reducing chronic intestinal inflammation in humans are limited. The colonic antigen used in at least one aspect of the invention may be any colonic antigen associated with UC. Previous studies have demonstrated the presence of specific autoantibodies in patients with UC against p40, a putative colonic autoantigen in UC, belonging to the tropomyosins family (Das, J. Immunol. 150:2487 (1993)). Studies have examined how the autoantibody is reactive against certain human tropomyosins, such as hTM5 (isoform 5).

Although patients with UC have autoantibody against hTM5, it is unlikely that the protective colon epithelial antigen will be hTM5. The hTM5 isoform is present in both the human colon and small intestine epithelial cell. The colon epithelium-specific antigen, however, is not present in the small intestine. The antigen has been detected specifically in colon epithelial cells, including LS-180 cells, yet is lacking in small intestine enterocytes (Das et al., Clin. Exp. Immunol. 118:219-27 ((1999). And as indicated in one aspect of the invention, the small intestine group, NSE-TNBS, failed to exhibit signs of tolerance. Furthermore, tropomyosin's molecular weight, depending on the isoform, ranges from approximately 30 to 42 kilodalton. In contrast, the CEP protein, as detected by the monoclonal antibody, is more than 200-kilodalton molecular weight. CEP is heavily glycosylated and is a membrane associated protein. A colon epithelium-specific hTM isoform with partial homology to hTM5, however, may be involved.

In a preferred embodiment, the oral tolerance may stem from a colon epithelial protein such as glycoprotein (ME>200 kd) reactive to the monoclonal antibody 7E₁₂H₁₂ (I9M isotype). Das et al., Production and Characterization of Monoclonal Antibodies to a Human Colonic Antigen Associated with Ulcerative Colitis Cellular Localization of the Antigen by Using the Monoclonal Antibody, J. Immuno. Vol. 139: 77-84. The monoclonal antibody 7E₁₂H₁₂ is reactive against extracolonic organs commonly involved in UC. Immunocytochemical studies using the 7E₁₂H₁₂ antibody demonstrated specific recognition of the colonic epithelial cells and not with 13 other epithelial organs, including other parts of the gastrointestinal tract and small intestine (Das, U.S. Pat. No. 5,869,048). This colon epithelial specific protein is mainly localized in the cell membrane.

Polypeptides

In one aspect, At least one aspect of the invention relates to human CEP polypeptides (or CEP proteins). The CEP polypeptides may be in the form of the “mature” protein or may be a part of a larger protein such as a fusion protein. However, fragments of the CEP polypeptides are also included in the invention. A fragment is a polypeptide having an amino acid sequence that entirely is the same as part, but not all, of the amino acid sequence of the aforementioned CEP polypeptides. Preferred fragments include, for example, truncation polypeptides having the amino acid sequence of CEP polypeptides, except for deletion of a continuous series of residues that includes the amino terminus, or a continuous series of residues that includes the carboxyl terminus or deletion of two continuous series of residues, one including the amino terminus and one including the carboxyl terminus. Other preferred fragments are biologically active fragments. Biologically active fragments are those that mediate CEP activity, including those with a similar activity or an improved activity, or with a decreased undesirable activity. Especially included are those that are antigenic or immunogenic in an animal, especially in a human.

Preferably, all of these polypeptide fragments retain the biological activity of the CEP protein, including antigenic activity. Variants of the defined sequence and fragments also form part of At least one aspect of the invention. Preferred variants are those that vary from the referents by conservative amino acid substitutions.

The CEP proteins, polypeptides and fragments of the invention can be prepared in any suitable manner. For instance, the CEP protein may be isolated, using methods well know in the art, and analyzed according to standard procedures to determine its amino-acid composition. For example, one simple method for isolating the CEP protein involves the use of immunologically-specific antibodies, such as the 7E₁₂H₁₂ monoclonal antibody, that can be used to precipitate the CEP protein. In another method, the CEP protein can be bound to beads that can be easily purified. Such beads can be magnetized, or simply dense enough to be separated form the non-associated protein by centrifugation. The CEP protein may then be subjected to amino acid composition, amino acid sequence, or protein concentration analysis according to known methods.

Using appropriate amino acid sequence information, synthetic CEP proteins of At least one aspect of the invention may be prepared by various synthetic methods of peptide synthesis via condensation of one or more amino acid residues, in accordance with conventional peptide synthesis methods. Preferably, peptides are synthesized according to standard solid-phase methodologies, such as may be performed on an Applied Biosystems Model 430A peptide synthesizer (Applied Biosystems, Foster City, Calif.), according to manufacturer's instructions. Other methods of synthesizing peptides or peptidomimetics, either by solid phase methodologies or in liquid phase, are well known to those skilled in the art. If produced in situ, the polypeptides may be purified from appropriate sources, e.g., appropriate vertebrate cells e.g., mammalian cells for instance cells from human, mouse, bovine or rat.

Alternatively, the availability of nucleic acid molecules encoding the polypeptides enables production of the proteins using in vitro expression methods known in the art. For example, a CEP cDNA or gene may be identified, isolated and cloned into an appropriate in vitro transcription vector, for in vitro transcription, followed by cell-free translation in a suitable cell-free translation system. In vitro transcription and translation systems are commercially available, e.g., from Promega Biotech, Madison, Wis., or BRL, Rockville, Md. While in vitro transcription and translation is not the method of choice for preparing large quantities of the protein, it is ideal for preparing small amounts of native or mutant proteins for research purposes, particularly since it allows the incorporation of radioactive nucleotides.

According to a preferred embodiment, larger quantities of CEP encoded polypeptide may be produced by expression in a suitable procaryotic or eucaryotic system. For example, part or all of a CEP DNA molecule may be identified, isolated and inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, and are positioned in such a manner as to permit expression of the DNA into the host cell. Such regulatory elements required for expression include promoter sequences, transcription initiation sequences and, optionally, enhancer sequences.

It is often advantageous to include an additional amino acid sequence that contain secretory or leader sequences, pro-sequences, sequences which aid in purification such as multiple histidine residues, or an additional sequence for stability during recombinant production. Hence, secretion signals may be used to facilitate purification of the resulting protein. The coding sequence for the secretion peptide is operably linked to the 5′ end of the coding sequence for the protein, and this hybrid nucleic acid molecule is inserted into a plasmid adapted to express the protein in the host cell of choice. Plasmids specifically designed to express and secrete foreign proteins are available from commercial sources. For example, if expression and secretion is desired in E. coli, commonly used plasmids include pTrcPPA (Pharmacia); pPROK-C and pKK233-2 (Clontech); and pNH8a, pNH16a, pcDNAII and pAX (Stratagene), among others.

The CEP proteins produced by in vitro transcription and translation or by gene expression in a recombinant procaryotic or eucaryotic system may be purified according to methods known in the art. Recombinant proteins can be purified by affinity separation, such as by immunological interaction with antibodies that bind specifically to the recombinant protein or fusion proteins such as His tags, as described below. Skilled practitioners commonly use such methods.

As mentioned, the proteins can be produced and fused to a “tag” protein in order to facilitate subsequent purification. These fusion proteins are produced by operably-linking the nucleic acid coding sequence of the “tag” protein to the coding sequence of the protein of interest, and expressing the fused protein by standard methods. Systems are commercially available that comprise a plasmid containing an expression cassette with the “tag” protein coding sequence and a polylinker into which a coding sequence of interest can be operably ligated. These fusion protein systems further provide chromatography matrices or beads that specifically bind the “tag” protein thereby facilitating the fusion protein purification. These fusion protein systems often have the recognition sequence of a protease at or near the junction of the “tag” protein and the protein of interest so that the “tag” protein can be removed if desired. Fusion protein systems include, but are not limited to, the His-6-tag system (Quiagen) and the glutathione-S-transferase system (Pharmacia).

Administration of CEP Composition for Therapeutic Purposes

The therapeutic component of At least one aspect of the invention is directed to a method for treating IBD in a subject, specifically UC and/or Crohn's disease which comprises administering a therapeutically effective composition of either colonic epithelial cellular protein, human colon tumor cell extract (specifically LS-180 cells), normal colon epithelial cell extract or T lymphocytes from subjects fed with colonic antigen extract, or a mixture thereof, to a subject.

CEP is effective in treating severe inflammation in the colon of animals by inducing tolerance, as indicated by the data expressed in Table 3 and FIGS. 1-8. Specifically, the administration of human colon extract and normal rat colon extract significantly reduces inflammation, whereas introducing no treatment, human fibroblast cellular extract, or normal rat small intestine results in severe inflammation and diseased tissue. CEP induces oral tolerance, and human colon epithelial cells such as LS-180 colon cancer cells may also induce such oral tolerance. Thus, the protective antigen(s) are organ (colon) specific.

In another embodiment of the invention, the treatment of a subject may involve the passive transfer of oral tolerance by administering T lymphocytes of animals fed with CEP. The reduction of inflammation may be mediated by distinct subsets of lymphocytes, including T lymphocytes from mesenteric lymph nodes and spleen of animals.

After obtaining the tolerance-inducing antigen, the antigen composition, or tolerogenic fragments thereof, is administered to a subject in accordance with At least one aspect of the invention. The composition may be administered orally, enterally, intranasaly, parenterally, subcutaneously, transdermally, intratumorally, rectally, intra-arterially, intramuscularly, by rapid infusion, nasopharyngeal absorption, intranasopharangeally, or by dermoabsorption. The composition may be administered in liquid, solid, or semi-solid form.

In a preferred embodiment, the antigen composition or fragment thereof is administered orally. Each oral composition according to At least one aspect of the invention may additionally comprise inert constituents including pharmaceutically acceptable carriers, dilutents, fillers, wetting agents, suspending agents, solubilizing or emulsifying agents, salts, flavoring agents, sweeteners, aroma ingredients or combinations thereof, as is well-known in the art. Liquid dosage forms may include a liposome solution containing the liquid dosage form. As known by those skilled in the art, suitable forms suspending liposomes include emulsions, pastes, granules, compact or instantized powders, suspensions, solutions, syrups, and elixirs containing inert dilutents, such as purified water.

Tablets or capsules may be formulated in accordance with conventional procedures employing solid carriers well known in the art. For example, a pharmaceutical preparation may contain an antigen composition dissolved in the form of a starch capsule, or hard or soft gelatin capsule, which is coated with one or several polymer films. Coating the external capsule wall with a polymer film prevents undesired dissolution of the capsule shell in the area of the stomach or upper small intestine. The choice and usage of appropriate polymers, including additional materials such as softeners and pore-forming agents, control the site of dissolution of the capsule and the release of solution containing the active agent.

Preparation of the composition may also include dissolving the composition in a solvent, which is suitable for encapsulation into starch or gelatin capsules, or in a mixture of several solvents and optionally solubilizers and/or other excipients. The solution is then filled into starch capsules, or hard or soft gelatin capsules in a measured dose, the capsules are sealed, and the capsules are coated with a solution or dispersion of a polymer or polymer mixture and dried. The coating procedure may be repeated once or several times.

The solvents that are appropriate for dissolving the active agent are those that are pharmaceutically acceptable and in which the composition dissolves. Examples of these are ethanol, 1,2-propylene glycol, glycerol, polyethylene glycol 300/400, benzyl alcohol, medium-chained triglycerides and vegetable oils.

Furthermore, medicament excipients may be added to the solution. Examples of such excipients are mono-/di-fatty acid glycerides, sorbitan fatty acid esters, polysorbates, lecithin, sodium lauryl sulphate, sodium dioctylsulphosuccinate, aerosil and water-soluble cellulose derivatives. Mixtures of solvents and excipients may also be used.

The soft or hard gelatin capsule may be coated with one or several polymer films, whereby the targeted capsule dissolution and release of the therapeutically effective composition is achieved through the film composition. The polymer or a mixture of polymers is dissolved or dispersed in an organic solvent or in a solvent mixture. For example, solvents include ethanol, isopropanol, n-propanol, acetone, ethyl acetate, methyl ethyl ketone, methanol, methylene chloride, propylene glycol monomethyl ether and water.

The properties of the polymer films may be further influenced by additions of pore-forming agents and softeners. Suitable pore-forming agents to form open pores, and thus to increase the diffusion rate through the polymer coating, are water-soluble substances, including lactose, saccharose, sorbitol, mannitol, glycerol, polyethylene glycol, 1,2-propylene glycol, hydroxypropyl cellulose, hydroxypropyl methylcellulose, methylcellulose, as well as mixtures thereof. Softeners include alkyl esters of citric acid, tartaric acid and 1,8-octanedi-carboxylic acid, triethyl citrate, tributyl citrate, acetyl triethyl citrate, dibutyl tartrate, diethyl sebacate, dimethyl phthalate, diethyl phthalate, dioctyl phthalate, castor oil, sesame oil, acetylated fatty acid glycerides, glycerol triacetate, glycerol diacetate, glycerol, 1,2-propylene glycol, polyethylene glycols and polyoxyethylene-polypropylene block copolymers, PEG-400 stearate, sorbitan monooleate, and PEG-sorbitan mono-oleate.

Additionally, parenteral administration of compositions of colonic antigen or fragment thereof may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate.

Dosages of the tolerance inducing substance for humans will vary, depending on factors such as half-life of the substance, potency, route of administration, and the condition of the patient. The patient's age, condition, sex, and extent of the disease and other variables that may be taken into account.

Furthermore, a colonic antigen composition should be administered in such way that it is present at a sufficient concentration to adequately provide protection. A lower concentration of the antigen treatment may be preferable for a longer period of time, or a higher concentration for a shorter period of time. Administration of an effective dosage may be in a single dose form or in multiple dosage forms. The dosage may be provided with an enteric coating and/or a sustained release mechanism, such as a degradable matrix or a reservoir.

The foregoing descriptions of embodiments of the invention are intended to be illustrative of the embodiments of At least one aspect of the invention, and are not intended to limit the invention in any way. Although the invention has been described with respect to specific modifications, the details thereof are not to be construed as limitations, for it will be apparent that various equivalents, changes and modifications may be resorted to without departing from the spirit and scope thereof and it is understood that such equivalent embodiments are to be included herein. All publications and patent applications are incorporated by reference as if fully set forth herein. At least one aspect of the invention is further illustrated by the following examples, which are not intended to limit the effective scope of the claims.

### EXAMPLE 1

### Effects of Different Extracts on the Rat Hapten-Induced Model of Colitis

Preparation of extracts for feeding: The colon and small intestine of normal homologous rats were cut into small pieces. The pieces were extensively washed, homogenized in PBS (phosphate-buffered saline solution) (pH 7.4), sonicated, and centrifuged for 10 minutes at 1000 g to remove large particles. The pieces were used as extracts from rat colon and small intestine for feeding.

Human colon epithelial cell extract and fibroblast cell extract were also prepared for feeding. Human colon cancer cells, LS-180, were cultured in Dulbecco's minimum essential medium (Sigma, St. Louis, Mo.) with 10% fetal calf serum for 4 to 5 days. The cells were harvested by trypsinization, washed with PBS, counted, and sonicated for 1 minute on ice. The total protein concentration in the lysate was measured with the Bio-Rad Protein assay kit (Bio-Rad Laboratories, Hercules, Calif.). A human fibroblast cell line, HT-1080, was used in parallel with a protocol identical to that used with LS-180 cells.

Animal models: Virgin female Sprague-Dawley rats were purchased from Harlan Labs, Inc. (San Diego, Calif.). Experimental groups consisted of at least 6 rats in each group.

Induction of oral tolerance: The Sprague-Dawley rats were placed in groups and orally fed 500 4 g/rat of tissue-extracted proteins in PBS from LS-180 human colon cancer cells (LSE group), HT-1080 human fibroblast cells (HTE group), homologous normal rat colon (NCE group), or small intestine (NSE group). (Table 1). The rats were fed every other day over a period of 9 days (total 5 times) with a number 20 gauge gastric feeding needle (Popper and Sons).

After 7 days of rest after the feedings, the rats were injected with TNBS. Using the rectally inserted tube, the rats in each group were intra-rectally administered TNBS (30 mg/rat) in 50% ethanol. A number 20 gauge metal feeding tube was inserted rectally into the colon of the rats. The tip of the tube was 8 cm proximal to the anus, approximately at the splenic flexure area. Hence, with the addition of TNBS, the groups were LSE-TNBS, HTE-TNBS, NCE-TNBS, and NSE-TNBS, respectively.

To examine the reproducibility, LS-180 cell extract was used on three different occasions in separate batches of rats (n=6 each time) followed by RE-TNBS. A separate parallel group included RE-TNBS only, without any feeding of proteins. (Table 1).

Assessment of colonic inflammation and damage: Rats were killed after 15 days for assessment of damage and for collection of tissue of blood. At the time the rats were killed, the colon was removed, opened by longitudinal incision. The colon was divided into two parts: 1) the distal colon, 0 to 8 cm from the anus to about the splenic flexure area; and 2) the proximal colon, >8 cm to the cecum. The two parts were weighed and examined. The tissue samples were examined with a stereomicroscope. Any visible damage was rated on a 0-5 scale, as developed by Morris et al., Gastroenterol. 96:795-803 (1989) (Table 2). The tissue samples from distal and proximal colon were longitudinally divided, and half of the specimens were frozen in dry ice immediately and stored at −80° C. The remaining halves were fixed in formalin by jellyroll technique and processed for paraffin block. Sections of the tissue samples were stained with hematoxylin and eosin.

A single pathologist (P.S.A.) performed a blinded fashion histologic assessment by light microscopy on coded slides. The colon wall thickness was determined separately on the hematoxylin-and-eosin stained sections by measuring the distance from the serosal surface to the luminal surface of the mucosal layer with a light microscope fitted with a measuring scale.

The small intestine processed separately for routine histology, and part of the small intestine was frozen. Myeloperoxidase (“MPO”) activity in the distal and proximal colons and small intestine was measured using a modified technique of Bradley with 4-aminoantipyrine as substrate. MPO activity is expressed as unit per milligram of protein.

Statistical Analysis: To examine the reproducibility, tests in the LSE-TNBS group (6 rats each time) and the RE-TNBS group (6 to 8 rats each time) were repeated on three occasions. Parallel groups were given rectal TNBS without oral feeding of tissue proteins. There was clearly a significant (P<0.001) protection in the macroscopic, ulcer, adhesion scores, and the histologic score (FIGS. 1A-B and 2A-B).

All results of the colon wet weight (in grams) of the different groups of rats are expressed as means±SEM (FIG. 3). The P values were measured against the RE-TNBS group, the rats that were given only rectal TN-BS.

The disease scores in the different groups of rats were measured on a scale of 0 to 5, with 5 being the most severe (FIG. 4). The P values were measured against the RE-TNBS group, the rats that were given only rectal TN-BS.

The results further indicate the thickness (in micrograms) of distal colon wall from luminal surface to serosa in the different groups of rats (FIG. 5). The P values were measured against the RE-TNBS group.

The MPO activity is also measured in MPO units (10⁻³) per milligram of total tissue protein from the distal colon in the different groups of rats (FIG. 6). The MPO activity measured inflammatory cellular infiltrate in the distal colon, proximal colon, and small intestine. The results are expressed as means±SEM. The P values were measured against the RE-TNBS group. The mean±SEM MPO activity in the proximal colon in the control group was 7.9±2.9 U×10⁻³/mg protein. There was not a significant difference in the values in the proximal colon and small intestine in all the other different rat groups from the control group. However, the MPO activities in the distal colon varied depending on each group (FIG. 6). The MPO values in the LSE-TNBS group were examined on three different occasions with three different groups of rats, and the results were highly reproducible.

Control Group

The disease score of the control rats was zero, with zero being the least severe (FIG. 4 and Table 3). The control group of rats had a low mean weight of the distal colon (P<0.0005). There was a significant (P<0.00004) increase in thickness in the RE-TNBS group when compared with the results in the control rats, shown in FIG. 5. The control rats indicated a thickness score of 500 micrograms. The mean±SEM MPO activity in the distal colon was 18.1±4.5 U×10⁻³/mg protein (P<0.001) (FIG. 6).

RE-TNBS Group

As shown in Table 3, 82% of the surviving rats in the RE-TNBS group showed consistent evidence of severe colitis in the distal colon. The proximal colon and entire small intestine, however, were both normal. FIG. 1A shows severe gross morphological damages with edema, ulcerations, thickening, and adhesions with adjacent organs of the rectosigmoid colon in a rat that received rectal TNBS. Ulceration and extensive inflammatory cell infiltration with polymorphonuclear cells, lymphocytes, eosinophils, and macrophages were quite apparent, as seen in FIG. 2A, displaying the histology of the tissue. In FIG. 3, the mean±SEM wet weight of the distal colon in the RE-TNBS group was 5.3±0.98 g, which is five-fold higher (P<0.0005) than that of the distal colon of normal control rats (0.98±0.15 g). Further, there was a significant mortality rate of 26% (Table 3). Rats treated with TNBS alone had significant disease, as indicated in FIG. 4, with a mean score of 4.5. Thickening of the wall with fibromuscular hypertrophy also occurred, and as shown in FIG. 5, the group experienced a thickness of 2750 micrograms of the distal colon wall. The mean±SEM MPO activity was 53.4±7.5 U×10⁻³/mg protein, which is a significant increase in MPO activity in the distal colon. (FIG. 6).

NCE-TNBS Group

Rats in the group fed with normal rat colon extract experienced a low disease score of approximately 1.4 (P<0.001), as shown by Table 3 and FIG. 4. The low mean±SEM wet weight of the distal colon in the NCE-TNBS group was 2.3±0-Ig (P<0.007) (FIG. 3). The thickness score in this group was not significantly different from the control group (P<0.0005) (FIG. 5). The mean±SEM MPO activity in the distal colon was 6.3±1.1 U×10⁻³/mg protein (P<0.0001) (FIG. 6).

NSE-TNBS Group.

The rats fed with rat small intestine had a significantly high disease score (P<0.2) as indicated in FIG. 4 and Table 3. The mean±SEM gross weight of the distal colon of the NSE-TNBS rats was 6.6±0.94 g, which was not significantly different from the rats fed only with TNBS (P<0.4) (FIG. 3). The thickness value of the colon wall was high (P<0.672) (FIG. 5). The rats in this group did not show any reduction in MPO activity in the distal colon when compared with the RE-TNBS group as seen in FIG. 6. The mean±SEM MPO activity was 161.1±44 U×10⁻³/mg protein (P<0.09) (FIG. 6), indicating high MPO activity, thus lessened protection of the distal colon.

LSE-TNBS Group

The disease score in the group of rats fed with LS-180 cell extract before TNBS enema was greatly reduced (P<0.001), shown in FIG. 4. As seen in FIG. 1B, rats that received LS-180 human colon cell extract had no inflammatory change. FIG. 2B further indicates the tissue is normal. There was also a low mortality rate of 5%, as seen in Table 3. The mean±SEM wet weight of the distal colon in the LSE-TNBS group was 2.2±0.3 g, which is significantly lower (P<0.008) than those in the RE-TNBS group (FIG. 3). FIG. 5 further indicates that the thickness of the distal colon wall in this group of rats was significantly lower than the group administered only TNBS (P<0.001). The thickness value was approximately 800 micrograms. When compared to the RE-TNBS group, the LSE-TNBS group had a significantly lower MPO activity value of approximately 18.1±4.5 U×10⁻³/mg protein (P<0.0001) (FIG. 6). The low MPO value indicates a significant suppression of inflammatory changes.

HTE-TNBS Group

Rats fed with extract of HT-1080 fibroblast cells had a significant disease score of approximately 4.5 on a scale from 0 to 5 (P<1) (FIG. 4, Table 3). The mean±SEM gross weight of the distal colon of the HTE-TNBS rats was 4.1±1.6 g, which was not significantly different from the rats fed only with TNBS (P<0.318) (FIG. 3). Five of the six rats in this group showed severe disease, as indicated in FIGS. 1C and 2C, as there were mucosal ulceration, edema, thickening of the wall, and severe inflammation in the rat colonic mucosa. The rats in this group experienced a thickening of the colon wall (P:<0.476), as shown in FIG. 5. The mean±SEM MPO activity in the distal colon was 39.8±2.1 U×10⁻³/mg protein (P<0.1) (FIG. 6), which is similar to the lesser protection seen in the RE-TNBS group.

### EXAMPLE 2

### Effect of Lymphocytes from Rats Administered LS-180 Cell Extract on the Rat Hapten-Induced Model

Passive transfer of lymphocytes: Mesenteric lymph nodes and spleen were removed from 4 rats that were fed with LS-180 cell extract five times over 9 days, as mentioned above. T lymphocytes were isolated with a T cell separation column for rats (Pierce, Rockford, Ill.) according to the manufacture's protocol. T cells (25×10⁴) from mesenteric lymph nodes or spleen were injected into naive rats (n=6 in each group) via tail vein in 200 μL PBS. (Table 1). After 7 days of rest, rats were challenged with TNBS by enema, as described above. The resulting groups were LS-L-TNBS (lymph nodes) and LS-S-TNBS (spleen) groups. With the identical protocol as used above, T lymphocytes from mesenteric lymph nodes and spleen of animals that were fed with HT-1080 fibroblast cell extract were injected into 8 naive rats (4 in each group with mesenteric lymph node T cells and splenic T cells, respectively.

The T lymphocytes from the mesenteric lymph nodes and the spleen of the rats fed with HT-1080 fibroblast cell extract were also passively transferred to eight rats, four rats in each group (HT-L-TNBS and HT-S-TNBS).

Statistical analysis: The passive transfer of T lymphocytes harvested from the mesenteric lymph nodes and spleen of rats fed with LS-180 resulted in clear protection, as indicated by the low disease scores in Table 3 and FIG. 4 (P<0.001 and P<0.03 for LS-L-TNBS and LS-S-TNBS, respectively). FIG. 2D shows minimal inflammatory changes in the tissue of a rat that received T lymphocytes in the LS-L-TNBS group. The LS-L-TNBS group also showed protection with mild disease (Table 3). The distal colon wet weight in the LS-L-TNBS and LS-S-TNBS groups were significantly lower than those in the RE-TNBS group (P<0.0008 and P<0.007, respectively) (FIG. 3). For both groups, FIG. 5 shows that the distal colon wall thickness also was significantly reduced when compared with the RE-TNBS values (P<0.001). The mean±SEM MPO activity in the distal colon for LS-L-TNBS and LS-S-TNBS were 9.0±3.9 U×10⁻³/mg protein and 6.8±2.5 U×10⁻³/mg protein, respectively (P<0.0001) (FIG. 6).

Of the three surviving rats from the HT-S-TNBS group had a disease score of 5, and one rat had a disease score of 4.

### EXAMPLE 3

### Correlation Between Serum Levels of Cytokines, IFN-γ and TGF-β and Oral Tolerance

Assaying in serum: Two important cytokines that have been implicated with inflammation of oral tolerance, IFN-γ and TGF-β were measured in serum. Both IFN-γ and TGF-β were assayed in serum by using the Quantikine ELISA Kit from R&D Systems (Minneapolis, Minn.). For the IFN-γ and TGF-β groups, the serum concentration was measured in micrograms per milliliter and picograms per milliliter, respectively.

Statistical analysis: High levels of IFN-γ were seen in the groups with severe inflammation, whereas lower levels were seen in the groups of rats with developed oral tolerance. In contrast, there is a positive correlation of TGF-β levels with protection. The P values were measured against the RE-TNBS group.

IFN-γ

In the RE-TNBS group, there was a significant increase in the serum IFN-γ levels (21.8±6 ˜μg/mL) compared to the control group (3.5±0.6 μg/mL, P<0.02) (FIG. 7). However, the rats fed with LS-180 extract (LS-TNBS group) had similar serum IFN-γ levels as the control group (3.4±0.5 μg/mL), and significantly lower than the rats administered only TNBS (P<0.016). Similarly, the rats fed with normal colon extracts (NCE-TNBS group) had lower IFN-γ values (0.5±0.5 ˜μg/mL, P<0.02). FIG. 7 further indicates that rats in the HTE-TNBS and NSE-TNBS groups had higher IFN-γ levels, similar to the RE-TNBS group (16.5±4.3 μg/mL and 17.1±5.5 μg/mL, P<0.53, P<0.39, respectively). As for the rats that received T lymphocytes (LS-L-TNBS and LS-S-TNBS groups), the IFN-γ levels were similar to the control group and lower than the RE-TNBS group value (P<0.026 and P<0.036, respectively (FIG. 7).

TGF-β

As shown in FIG. 8, the serum level of TGF-β for the control group was 184.9±26.3 pg/mL (P<0.635). The RE-TNBS group value, however, Both the LSE-TNBS and NCE-TNBS groups showed protection (654±92.4 pg/mL and 518.8±67.7 pg/mL, respectively), and were significantly higher than the RE-TNBS group value (P<0.003 and P<0.0003, respectively). The lymphocyte groups, LS-L-TNBS and LS-S-TNBS, similarly have increased TGF-β levels (714.7±102.6 pg/mL and 673.4±82.5 pg/mL, respectively, P<0.002) when compared to levels in the RE-TNBS group.

On the other hand, the rats fed with HT-1080 had a lower TGF-β level (234±24.9 pg/mL) similar to that of the RE-TNBS group (P<0.259) (FIG. 8). However, the animals fed with small intestinal extract had a high TGF-β level (680±95.4 pg/mL). It is unclear whether this high level is due to severe disease causing TGF-β secretion from a non-lymphocyte source or is a different form of TGF-β that cannot be differentiated by the assay used.

All references cited herein are incorporated by reference in their entirety. The descriptions in the present invention are provided only as examples and should not be understood to be limiting on the claims.

What is claimed is:

1. A composition for the treatment of inflammatory bowel disease comprising a CEP wherein the CEP is a glycoprotein with a molecular weight over 200 kD and reactive to a monoclonal antibody 7E₁₂H₁₂.

2. The composition of claim 1, wherein the inflammatory bowel disease is ulcerative colitis or Crohn's disease.

3. A composition for the treatment of inflammatory bowel disease comprising a human colon cancer cell extract.

4. The composition of claim 3, wherein said human colon cancer cell extract causes the induction of tolerance.

5. The composition of claim 3, wherein said human colon cancer cell extract comprises a LS-180 cells extract.

6. A composition for the treatment of inflammatory bowel disease comprising a colon epithelial cell extract.

7. The composition of claim 6, wherein said colon epithelial cell extract causes the induction of tolerance.

8. A composition for the treatment of inflammatory bowel disease comprising T lymphocytes extracted from a subject that has been fed with a colon epithelial cell extract.

9. The composition of claim 8, wherein said T lymphocytes causes the induction of tolerance.

10. The composition of claim 8 wherein the T lymphocytes are from a spleen or mesenteric lymph nodes of the subject

11. A method for treating inflammatory bowel disease comprising administering T lymphocytes from a subject fed with a colon epithelial cell extract.

12. The method of claim 11, wherein administering the T lymphocytes induces tolerance.

13. The method of claim 11, wherein the subject is a human.

14. The method of claim 11, wherein administering the T lymphocytes induces a cytokine to reduce inflammation.

15. The method of claim 14, wherein the cytokine is TGF-β

16. The method of claim 11, wherein administering the T lymphocytes induces a suppressor T cell response.

17. The method of claim 11, wherein the extract is a LS-180 human colon cancer cell extract, or normal colon cell extract.

18. The method of claim 11, wherein the T lymphocytes are administered orally, enterally, intranasaly, peritoneally, subcutaneously, transdermally, intratumorally, rectally, intra-arterially, intramuscularly, by rapid infusion, nasopharyngeal absorption, intranasopharangeally, or by dermoabsorption.

19. The method of claim 14, wherein the T lymphocytes are from mesenteric lymph nodes or a spleen.

20. A method for reducing inflammation comprising administering to a subject a therapeutically effective amount of at least a composition selected form the group consisting of the CEP of claim 1, a colon epithelial cell extract, a LS-180 human colon cancer cell extract, a normal colon cell extract and T lymphocytes of a subject fed with the colon epithelial cell extract.

21. The method of claim 20, wherein said administration of the composition results in the induction of TGF-β.

22. The method of claim 20, wherein said administration results in the reduction of inflammation.

23. The method of claim 22, wherein said administration causes a Th2 CD4 T cell response.

24. The method of claim 20, wherein said composition is administered orally, enterally, intranasaly, parenteral injection, subcutaneously, transdermally, intratumorally, rectally, intra-arterially, intramuscularly, by rapid infusion, nasopharyngeal absorption, intranasopharangeally, or by dermoabsorption.

25. A composition for the treatment of inflammatory bowel disease comprising at least one member selected from the group consisting of the CEP of claim 1, a colon epithelial cell extract, a LS-180 human colon cancer cell extract, a normal colon cell extract and T lymphocytes of a subject fed with the colon epithelial cell extract.

26. The composition of claim 25, wherein the inflammatory bowel disease is ulcerative colitis.

27. The composition of claim 25, wherein the inflammatory bowel disease is Crohn's disease.

28. A method for treating inflammatory bowel disease comprising administering to a subject a therapeutically effective amount of at least a composition selected form the group consisting of the CEP of claim 1, a colon epithelial cell extract, a LS-180 human colon cancer cell extract, a normal colon cell extract and T lymphocytes of a subject fed with the colon epithelial cell extract.

29. The method of claim 28, wherein said inflammatory bowel disease is ulcerative colitis.

30. The method of claim 28, wherein said inflammatory bowel disease is Crohn's disease.

31. The method of claim 28, wherein the administration of the composition induces tolerance.

32. The method of claim 28, wherein the subject is a human.

33. The method of claim 28, wherein administering the composition induces a cytokine to reduce inflammation.

34. The method of claim 33, wherein the cytokine is TGF-β.

35. The method of claim 28, wherein administering the composition induces a suppressor T cell response.

36. The method of claim 28, wherein the composition is administered orally, enterally, intranasaly, peritoneally, subcutaneously, transdermally, intratumorally, rectally, intra-arterially, intramuscularly, by rapid infusion, nasopharyngeal absorption, intranasopharangeally, or by dermoabsorption.

37. The method of claim 28, wherein the composition is administered in a liquid, solid, or semi-sold form.

38. The method of claim 28, wherein the composition further comprises a pharmaceutically acceptable carrier.

39. The method of claim 28, wherein the composition further comprises dilutents, fillers, wetting agents, suspending agents, solubilizing agents, emulsifying agents, salts, flavoring agents, sweeteners, aroma ingredients or combinations thereof.

40. The method of claim 28, wherein the composition further comprises emulsions, pastes, granules, compact powders, instantized powders, suspensions, solutions, syrups, or elixirs containing inert dilutents.
